(19) United States
(12) Patent Application Publication
(10) Pub. No.: US 2021/0003559 A1
(43) Pub. Date: Jan. 7, 2021

(54) Method for Assessing a Compound Interacting with a Target on Epithelial Cells

(71) Applicants: Hoffmann-La Roche Inc., Little Falls, NJ (US); F. Hoffmann-La Roche AG, Basel (CH); Emulate, Inc., Boston, MA (US)

(72) Inventors: Cristina Bertinetti-Lapatki, Basel (CH); Lauriane Cabon, Basel (CH); Adrian B. Roth, Basel (CH); Annie Moisan, Basel (CH); Nikolche Gjorevski, Basel (CH); Jordan S. Kerns, Boston, MA (US); Geraldine A. Hamilton, Boston, MA (US); Catherine Karalis, Boston, MA (US); Heather Grant, Boston, MA (US); Riccardo Barrile, Boston, MA (US); Debora Barreiros Petropolis, Boston, MA (US); Chaitra Belgur, Boston, MA (US)

(21) Appl. No.: 16/912,391

(22) Filed: Jun. 25, 2020

(30) Foreign Application Priority Data
Jul. 2, 2019 (EP) 19183985.1

(51) Int. Cl. G01N 33/50

(52) U.S. Cl. CPC G01N 33/5044

(57) ABSTRACT

Disclosed herein is a method for assessing a compound interacting with a target on polarized epithelial cells. The method comprising the steps of providing an organ chip comprising a main channel and polarized epithelial cells, wherein the main channel is divided into an apical channel and a basal channel separated by the polarized epithelial cells, wherein the apical side of the polarized epithelial cells is directed towards the apical channel and the basolateral side of the polarized epithelial cells is directed towards the basal channel. Determining the localization and optionally the expression level of the target on the polarized epithelial cells. Administering the compound and optionally immune cells, preferably peripheral blood mononuclear cells (PBMC) to the basal channel, when the target is localized on the basolateral side of the epithelial cells or administering the compound and optionally immune cells, preferably peripheral blood mononuclear cells (PBMC) to the apical channel, when the target is localized on the apical side of the epithelial cells. Measuring a parameter of the administration of the compound and the peripheral blood mononuclear cells.

### CROSS REFERENCE TO RELATED APPLICATION

This application claims priority to European Patent Application No. 19183985.1 filed Jul. 2, 2019, the disclosure of which is hereby incorporated by reference in its entirety.

### FIELD OF DISCLOSURE

The present invention lies in the technical field of organ on a chip technology including intestine and lung chips. In particular, the invention relates to a method for assessing a compound interacting with a target on epithelial cells.

### Background

While animal testing is still a crucial and widely used tool for the assessment of the characteristics of a lead compound in the pharmaceutical industry, its use is subject to debate, not only for ethical reasons, but also for the quality and information value of the data obtained for the assessment of a potential human drug. In particular, the human and rodent immune response often shows considerable differences. Thus, various alternatives have been developed aiming towards obtaining data which is more reliable and more significant for the human organism. The two major alternative tools include advanced computational simulations (in silico) and cell cultures (in vitro).

Compounds for treating cancer, which may for example include small molecules or antibodies, are often designed to interact with specific targets which are ideally only expressed on the tumor cells, but not on healthy cells. However, it is often the case that also healthy cells express these targets, albeit at lower levels. As a result, the interaction of the lead compound and its associated potential toxicity for the healthy cells has to be assessed at a preclinical state. For determining these liabilities in vitro, advanced models of human organs have been developed. Amongst these, classical 2D monolayer cell cultures enable to mimic the delivery of the lead compound to the cell surface exposed to the environment. A wide variety of human cell types can be used in these cultures, such as intestine, lung, heart, liver cells and others.

Human organ-on-a-chip platforms combine microscale engineering technologies with human cell culture to recapitulate key physiological and functional aspects of whole living organs. For example, WO 2018 102 202, whose content is introduced herein by reference, discloses an intestine on a chip.

### SUMMARY OF DISCLOSURE

While 2D monolayer cultures are suitable for assessing the effects of a lead compound which is directly exposed to the cell surface, it is not possible to investigate or even differentiate between other routes of administration. For example, in 2D monolayer cultures, epithelial cells often lose their polarization and may thus express both apical and basolateral markers at the cell surface. Consequently, the results obtained from 2D monolayer cultures are often not suited as accurate models for the human organs, such as the gut or the lung. Thus, such models do not reliably allow for the prediction of toxic effects of a compound being delivered by a particular route of delivery. For example, if a specific target was to be expressed on the basolateral side of the epithelial cells, a 2D monolayer model in which the epithelial cells lost their polarization or lost their basolateral expression could predict that a particular systemically delivered compound is safe, as no toxic side effects would be observed. However, in reality, due to loss of basolateral expression and due to the integrity of the intestinal or pulmonary barrier, the lead compound may not be able to penetrate the barrier, and will thus not reach the target in the 2D model. If the lead compound is however administrated in a subject via the systemic pathway, it may interact with a target expressed on the basolateral side of a healthy cell and thus in fact show toxicity.

In general, transwell platforms in theory permit apical and basal access, but are static systems in which the dynamic movement of body fluids is absent. For example, immune cells, antibodies and/or lead compounds may sediment and thus fail to infiltrate the epithelium.

A particular interesting class of compounds for cancer immunotherapy are T-cell bispecific antibodies (TCBs). TCBs exert their anti-tumor activity through simultaneously binding to a cancer surface antigen and a T-cell receptor, thereby both activating the latter and physically crosslinking it to the target cells. This approach is particularly promising in targeting less immunogenic, neo-antigen-lacking tumors, as T-cells can be recruited and activated independently of the specificity of their surface receptors. The therapeutic potential of TCBs is exemplified by a number of molecules, directed against solid and blood tumors, which are currently in various stages of clinical evaluation. Preferably, the TCBs bind an epithelial target, which may be expressed on a cancer cell, and an immune cell, e.g. a T-cell receptor such as CD3.

However, because the cytotoxic potential of TCBs does not rely on the intrinsic receptor specificity of T-cells, and because the specific target antigens are often expressed at low levels in normal tissue, TCBs are subject to “off-tumor on-target” liabilities. For example, the wide application of therapies based on CD3 bispecific antibodies interacting with classical tumor antigens, including EpCAM and EGFR, has been hampered by target organ toxicities in humans and cynomolgus monkey. Assessment of safety concerns in the preclinical stage is therefore crucial for TCBs, and the fundamental discrepancies between human and rodent immune responses necessitate the development of advanced models of human organs for the evaluation of TCB pharmacology and related liabilities in vitro. Compared to other cancer agents, such as small molecules or standard antibodies, a crucial determinant of potential off-tumor TCB activity in the gut is the mutual accessibility of the antibody and its target antigen, which, in turn, is specified by i) the route of TCB administration, ii) the subcellular localization of the antigen and iii) the intestinal or pulmonary barrier integrity.

As mentioned above, classical 2D monolayer cell cultures cannot provide access to both the apical and the basolateral side of the epithelial cells. Thus, 2D models fail to capture both important determinants of TCB activity in the gut or the lung, cell and tissue surface localization of the antigen, and preferred route of TCB delivery.

As currently known methods are insufficient, it is an overall object of the invention to improve the state of the art regarding the assessment of a compound interacting with a target on polarized epithelial cells.

In some embodiments, a method is provided for assessing compound pharmacology and/or associated gut or lung tissue liabilities, preferably in a target expression and/or tissue localization dependent manner.

In preferred embodiments, a method is provided for assessing whether a compound induces deterioration and leakage of the intestinal or pulmonary barrier.

In preferred embodiments, a method is provided which mimics the native intestinal epithelium or the native pulmonary epithelium with higher fidelity than the prior art.

According to an aspect, the invention is directed to method for assessing a compound interacting with a target on polarized epithelial cells, in particular for assessing the safety of a compound. The method comprising the steps:
a. Providing an organ chip comprising a main channel and polarized epithelial cells. The main channel of the organ chip is divided into an apical channel and a basal channel, which are separated by the polarized epithelial cells. The apical side of the polarized epithelial cells is directed towards the apical channel and the basolateral side of the polarized epithelial cells is directed towards the basal channel;
b. Determining the localization and occurrence of the target on the polarized epithelial cells;
c. Administering the compound and optionally immune cells, preferably peripheral blood mononuclear cells (PBMC) to the basal channel, when the target is localized on the basolateral side of the epithelial cells;
   or
   Administering the compound and optionally immune cells, preferably peripheral blood mononuclear cells (PBMC) to the apical channel, when the target is localized on the apical side of the epithelial cells;
d. Measuring a parameter of the administration of the compound and the peripheral blood mononuclear cells.

The organ chip used in the method according to the invention may in some embodiments be an intestine chip as disclosed in Examples 1 and 2 and FIG. 2-6 of WO 2012 118 799 which is incorporated herein by reference. The organ chip may for example comprise a membrane, which also separates the basal channel and the apical channel. The membrane may carry the polarized epithelial cells and may be permeable for the compound to be assessed and/or for PBMCs. The membrane may be coated with materials promoting cell adhesion, such as collagen, laminin, fibronectin, glycoproteins, vitronection, elastins, fibrin, proteoglycans, heparin sulfate, chondroitin sulfate, keratin sulfate, hyaluronic acid, fibroin, chitosan or combinations thereof.

In step d. the measured parameter of the administration is typically a parameter associated with the administration, i.e. a parameter which is the result of said administration. In particular, the measured parameter of the administration is associated with the interaction of the compound with the target. Thus, the parameter may be a consequence of or result from said interaction. The skilled person understands that the term measured parameter of administration may be understood as a pharmacological parameter or as a safety parameter.

Typically, the polarized epithelial cells separating the apical and basal channel form a tight barrier, i.e. a barrier with same permeability than a native organ barrier, such as the native intestinal or native pulmonary barrier and provides a physical separation of the apical channel and the basal channel. Thus, typically only particles up to 4 Å in radius may migrate through tight junction pore pathways.

In certain embodiments, immune cells in step c. may be peripheral blood mononuclear cells, in particular CAR-T cells, i.e. genetically engineered T cells, expressing chimeric antigen receptors, wherein the chimeric antigen receptors are specific for tumor cells.

In some embodiments, the apical side of the polarized epithelial cells is arranged within the apical channel and/or the basolateral side of the epithelial cells is arranged within the basal channel.

Determining the occurrence of the target on the polarized epithelial cells can include the determination of expression levels of the target on the epithelial cells.

In some embodiments, the localization of the target within the native organ, particularly within the native lung or the native intestine is determined before or after step b.

In some embodiments, the organ chip is an intestine chip and the polarized epithelial cells are intestine epithelial cells. Particularly, the polarized epithelial cells may comprise Caco-2 or primary cells, i.e. primary human intestinal epithelial cells. In certain embodiments, the polarized epithelial cells comprise primary organoids.

In other embodiments, the organ chip is a lung chip and the polarized epithelial cells are alveolar epithelial cells primary cells, i.e. primary human pulmonary cells. In certain embodiments, the polarized epithelial cells comprise primary organoids.

In some embodiments of a lung chip, the basal channel comprises pulmonary microvascular endothelial cells. Typically, these may separate the basal channel from the apical channel. The pulmonary microvascular endothelial cells may be carried by the membrane separating the basal and the apical channel.

In embodiments of a lung chip wherein the basal channel comprises pulmonary microvascular endothelial cells, the target may also be expressed on the pulmonary microvascular endothelial cells. Thus, step b. comprises in this case determining the localization and optionally the expression level of the target on the pulmonary microvascular endothelial cells and step c. comprises administering the compound and optionally immune cells, preferably peripheral blood mononuclear cells (PBMC) to the basal channel, when the target is localized on the basolateral side of the pulmonary microvascular endothelial cells; or administering the compound and optionally immune cells, preferably peripheral blood mononuclear cells (PBMC) to the apical channel, when the target is localized on the apical side of the pulmonary microvascular endothelial cells. The apical side of the pulmonary microvascular endothelial cells is typically the side directed towards or into the apical channel and the basolateral side of the pulmonary microvascular endothelial cells is typically the side directed towards or into the basal channel.

In some embodiments, step b. is performed by immunofluorescence analysis. For example, the compound can be fluorescently labelled. If the compound is a TCB, the TCB or the PCMBs may be fluorescently labelled. Furthermore, the target may be labelled by a labeling antibody binding to the target. Alternatively, step b. may be performed by other spectroscopic methods, for example Raman spectroscopy. In this case, either the compound or the PCMBs are labelled with Raman active moieties, such as alkyne, dialkyne or phenyl-dialkyne.

In further embodiments step b. comprises the comparison of the localized targets on the polarized epithelial cells with the targets in a native organ, such as the native intestine or the native lung. This step is only necessary, if the organ chip has not been used and evaluated previously.

In some embodiments the compound is a compound which is to be administered via systemic circulation. The skilled person understands that such a compound represents a lead compound which is envisioned to be used as a medicament which is administered via systemic circulation.

In further embodiments the polarized epithelial cells and/or the basal channel and/or the apical channel is covered with an extracellular matrix (ECM), in particular a ECM hydrogel. Examples of ECM materials include Matrigel®, Cultrex®, ECM harvested from human donors or other species or collagen.

In some embodiments the target with which the compound interacts is an antigen, in particular a tumor-overexpressing protein. In some embodiments, the target may be located on the basolateral side of the epithelial cells. In other embodiments, the target may be located on the apical side of the epithelial cells.

In further embodiments the method comprises a first procedure and a second procedure. The first procedure comprises the steps a. to d. as described above, wherein in step c. the compound is administered to the basal channel. The second procedure comprises the steps:
i. Administering the compound and peripheral blood mononuclear cells to the apical channel;
ii. Measuring a parameter of the administration of the compound and optionally the peripheral blood mononuclear cells.

The skilled person understands that the terms first and second procedure do not imply a certain order of events. Thus, in some embodiments, the first procedure is carried out before the second procedure, while in other embodiments the second procedure is carried out before the first procedure.

In some embodiments the parameter measured in step d. and/or ii. is a safety parameter associated with a state of the polarized epithelial cells and/or with a state of a tight barrier formed by the polarized epithelial cells. Alternatively, or additionally, the parameter is a pharmacological parameter, i.e. efficacy, specificity, activity, availability, etc. It is understood that step b. and/or step ii. does not necessarily comprise the measurement of only a single parameter, but may also comprise the measurement of several parameters, particularly of both at least one pharmacological parameter and at least one safety parameter associated with the integrity or permeability of the epithelial barrier.

In some embodiments, the safety parameter is associated with the integrity or permeability of the barrier formed by the polarized epithelial cells and/or the pharmacological parameter is associated with side effects or potency of the compound, peripheral blood mononuclear cell activation or cytokine release.

In further embodiments, the flow rate of the administered compound and optionally the immune cells, through the apical channel or the basal channel is 10 to 60 μl/h, preferably 15 to 50 μl/h, more preferably 20 to 40 μl/h, most preferably essentially 30 μl/h.

In some embodiments, the compound interacting with the target on polarized epithelial cells is an antibody, or a bispecific antibody, in particular a T-cell bispecific antibody. Alternatively, the compound may be a small molecule. Preferably, the compound comprises a T-cell bispecific antibody and the target comprises an antigen. If the compound is a bispecific antibody, in particular a TCB, both the bispecific antibody, in particular the TCB, and PCMBs are administered in step c.

In further embodiments, the compound is a bispecific agent, binding an epithelial target and an immune cell. The bispecific agent is interacting with the target and comprises at least one additional binding site for binding a second target, preferably an antigen. The interaction may involve binding of the bispecific agent to the target or may involve a linker connecting the two. The target is preferably an epithelial target, including, among others, targets bound to and/or expressed on the surface of epithelial cells. In further preferred embodiments, the second target may be an antibody or a fluorescent label.

In further embodiments, the polarized epithelial cells comprise Caco-2 or primary cells, in particular primary human intestinal epithelial cells. In certain embodiments, the polarized epithelial cells comprise primary organoids.

In some embodiments the organ chip comprises a first and a second vacuum channel, wherein the main channel is arranged between the first and second vacuum channel, wherein peristaltic movement or breathing movement is modelled by the first and second channels during step c and/or step ii.

In some embodiments, steps a. to d. are performed with a first compound of interest and thereafter with a second compound of interest particularly on another organ chip as described herein, wherein the parameter of the first compound of interest and the parameter of the second compound of interest obtained in step d. are compared. After comparing the two parameters, the parameters may be interpreted. For example, when the parameter is a safety parameter indicating for example cytokine release or T cell activation, a higher cyctokine release or T cell activation may indicate a less favorable clinical safety profile. The parameter may represent a binding affinity of the compounds of interest in particular, if the compound of interest is an antibody.

The following numbered clauses describe exemplary aspects, embodiments, or examples of the present invention.
1. A method for assessing a compound interacting with a target on polarized epithelial cells, the method comprising the steps:
   a. providing an organ chip comprising a main channel and polarized epithelial cells, wherein the main channel is divided into an apical channel and a basal channel separated by the polarized epithelial cells, wherein the apical side of the polarized epithelial cells is directed towards the apical channel and the basolateral side of the polarized epithelial cells is directed towards the basal channel;
   b. determining the localization and optionally the expression level of the target on the polarized epithelial cells;
   c. administering the compound and optionally immune cells, preferably peripheral blood mononuclear cells (PBMC) to the basal channel, when the target is localized on the basolateral side of the epithelial cells;
      or
      administering the compound and optionally immune cells, preferably peripheral blood mononuclear cells (PBMC) to the apical channel, when the target is localized on the apical side of the epithelial cells;
   d. measuring a parameter of the administration of the compound and optionally the immune cells, preferably the peripheral blood mononuclear cells.
2. The method according to clause 1, wherein the organ chip is an intestine chip, wherein the polarized epithelial cells are intestine epithelial cells.
3. The method according to clause 2, wherein the polarized epithelial cells comprise Caco-2 or primary cells, in particular primary human intestinal epithelial cells.
4. The method according to clause 1, wherein the organ chip is a lung chip, wherein the polarized epithelial cells are alveolar epithelial cells or primary cells.
5. The method according to clause 4, wherein the basal channel comprises pulmonary microvascular endothelial cells.
6. The method according to any of the preceding clauses, wherein step b. is performed by immunofluorescence analysis.
7. The method according to any of the preceding clauses, wherein step b. comprises the comparison of the localized target on the polarized epithelial cells with the target in a native organ, particularly in the native intestine or in the native lung.
8. The method according to any of the preceding clauses, wherein the compound is a compound to be administered via systemic circulation.
9. The method according to any of the preceding clauses, wherein the polarized epithelial cells and/or the basal channel and/or the apical channel is covered with an extracellular matrix.
10. The method according to any of the preceding clauses, wherein the target on the polarized epithelial cells is an antigen, in particular a tumor-overexpressing protein.
11. The method according to any of the preceding clauses, wherein the method comprises a first procedure, wherein the first procedure comprises steps a. to d., wherein in step c. the compound is administrated to the basal channel, the method further comprising a second procedure, wherein the second procedure comprises the steps:
   i. administering the compound and peripheral blood mononuclear cells to the apical channel;
   ii. measuring a parameter of the administration of the compound and optionally the peripheral blood mononuclear cells.
12. The method according to any of the preceding clauses, wherein in step d. and/or step ii. the parameter is:
   a safety parameter associated with a state of the polarized epithelial cells and/or with a state of a tight barrier formed by the polarized epithelial cells; and/or
   pharmacological parameter.
13. The method according to clause 12, wherein the safety parameter is associated with the integrity or permeability of the barrier formed by the polarized epithelial cells and/or wherein the pharmacological parameter is associated with side effects or potency of the compound, peripheral blood mononuclear cell activation or cytokine release.
14. The method according to any of the preceding clauses, wherein the flow rate of the administered compound and optionally the immune cells, through the apical channel or the basal channel is 10 to 6 μl/h, preferably 15 to 50 μl/h, more preferably 20 to 40 μl/h, most preferably essentially 30 μl/h.
15. The method according to any of the preceding clauses, wherein the compound is an antibody, a bispecific antibody, in particular a T-cell bispecific antibody, or a small molecule.
16. The method according to clause 15, wherein the bispecific antibody, in particular the T-cell bispecific antibody, binds an epithelial target and an immune cell.
17. The method according to any of the preceding clauses, wherein the organ chip comprises a first and a second vacuum channel, wherein the main channel is arranged between the first and second vacuum channel, wherein peristaltic movement is modelled by the first and second channels during step c and/or step ii.
18. The method according to any of the preceding clauses, wherein steps a. to d. are performed with a first compound of interest and thereafter with a second compound of interest, wherein the parameter of the first compound of interest and the parameter of the second compound of interest obtained in step d. are compared.
19. The method according to clause 18, wherein the comparing is performed by a processor or computer in a computer-implemented method.
20. The method according to any of the preceding clauses, wherein any of the steps are performed at least in part, in a computer-implemented method using a computer or processor.

### BRIEF DESCRIPTION OF THE FIGURES

FIG. 1 shows a flow chart of a method according to an embodiment of the invention.

FIG. 2 shows a schematic cross-sectional view of an intestine chip as used in the embodiment of FIG. 1.

FIG. 3a shows pharmacologic data obtained by using a conventional 2D monolayer cell culture in combination with a TCB targeting STEAP1.

FIG. 3b shows pharmacologic data obtained by using a conventional 2D monolayer cell culture in combination with a TCB targeting CEA.

FIG. 4a shows pharmacologic data obtained by using the method according to an embodiment of the invention in combination with TCB targeting STEAP1.

FIG. 4b shows the trans-epithelial permeability of the epithelial cells used for obtaining the pharmacology obtained in FIG. 4a.

FIG. 5a shows pharmacologic data obtained by using the method according to an embodiment of the invention in combination with TCB targeting CEA.

FIG. 5b shows the trans-epithelial permeability of the epithelial cells used for obtaining the pharmacology obtained in FIG. 5a.

FIG. 6a shows the localization of CEA within a conventional 2D monolayer cell culture.

FIG. 6b shows the localization of CEA within an intestine chip as used in the present invention.

FIG. 6c shows the localization of STEAP1 within an intestine chip as used in the present invention.

FIG. 7a shows pharmacologic data obtained by using the method according to an embodiment of the invention using primary epithelial organoids in combination with TCB targeting CEA.

FIG. 7b shows the trans-epithelial permeability of the epithelial cells used for obtaining the pharmacology obtained in FIG. 7a.

FIG. 8 shows a comparison of pharmacological data obtained for two different CEA-targeting TCBs.

FIG. 9 shows a schematic cross-sectional view of a lung chip as according to another embodiment of the invention.

FIG. 10 shows FOLR1 expression in chip within a lung chip as used in the present invention.

FIG. 11 shows TCB-mediated cytotoxicity and cytokine release in one embodiment of a lung chip, treated with 20 μg/ml of FOLR1 high affinity antibody (FOLR1(Hi) TCB). Activated CD8+CD69+ T cells; apoptotic cells, and cytokine release are represented as granzyme B; IFNγ; TNFα; and IL-6. Nonbinding control antibody=NT. Upper two panels: One-way ANOVA, Tukey's Multiple Comparison Test, *p<0.05, n=4. Lower panels: 2-way ANOVA, Tukey's Multiple Comparison Test, ****p<0.0001, n=4.

FIG. 12 shows graphs representing T-Cell activation (top left graph); T-Cell (PBMC) attachment to the Alveolus Epithelium (top right graph, after 48 h of treatment); apoptotic cell quantification, lower left and right graphs, respectively, for an assay performed using a lung chip according to one embodiment. Middle right and lower panels demonstrate a selective increase in cytokine secretion between FOLR1(Hi) TCB over FOLR1(Lo) TCB at around 2 μg/ml up to 20 μg/ml.

### EXEMPLARY EMBODIMENTS

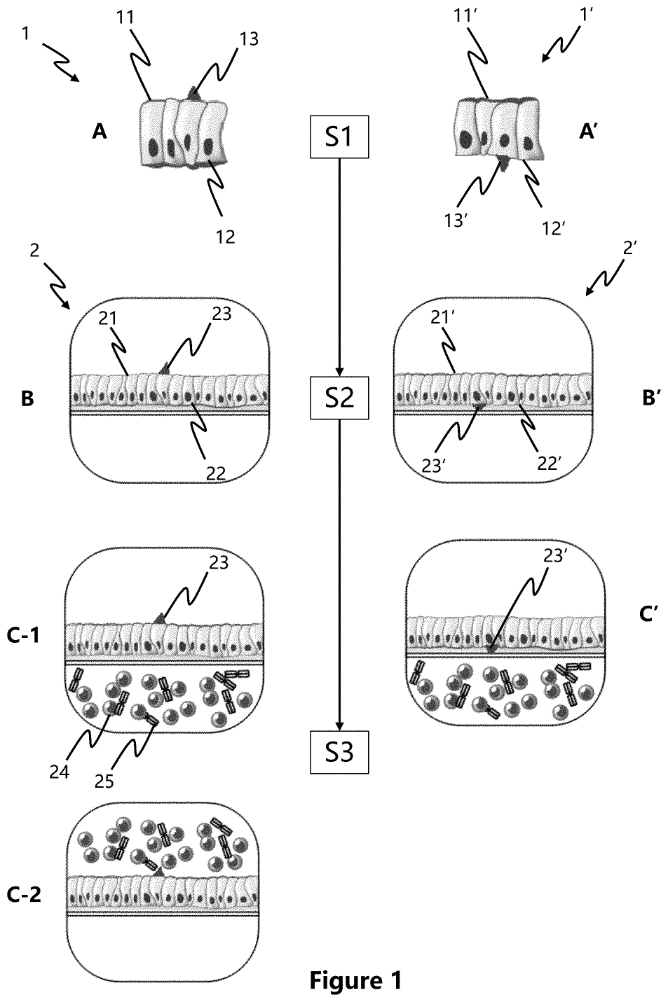

FIG. 1 shows an exemplary embodiment of a method according to the invention. In a first step S1, the location of target antigen 13, respectively 13′ is determined in the native intestine 1 and 1′. It is noted that only one exemplary antigen is schematically shown. The skilled person is well aware that more than one antigens may naturally be present. Optionally, the expression level of the antigen in the native intestine is determined. In scenario A, shown on the left of FIG. 1, antigen 13 is located on the apical side 11 and not on basolateral side 12 of the epithelial cells. In contrast, in scenario A′, which is illustrated on the right side of FIG. 1, antigen 13′ is located on the basolateral side 12′ of the epithelial cells. Step 1 is optional and only necessary if the localization of the target antigen within the native intestine is unknown or needs to be confirmed. In step S2, an intestine chip 2, respectively 2′, is provided. FIG. 1 shows the main channel of the intestine chip as a cross-sectional view. Furthermore, the localization of the target antigen 23, respectively 23′, on the epithelial cells within the intestine chip is determined and compared to the localization of the antigen within the native intestine 1, which has either been determined in step S1 or which is already known in the art. Concomitantly, the expression level of the antigen is determined and it is in general verified whether the expression level obtained reflects the expression level of the antigen in the native intestine. In scenario B, the target antigen 23 is located on the apical side 21 and not on the basolateral side 22 of the epithelial cells. In contrast, in scenario B′, target antigen 23′ is located on the basolateral side 22′.

Following the verification of physiological target expression, TCBs 25 and T-cells 24 are introduced into the basal channel or the apical channel in step S3. Furthermore, TCB-mediated pharmacological parameters and safety parameters are measured. In scenario C′, in which antigen 23′ is located on the basolateral side, TCBs 25 and T-cells 24 are introduced into the basal channel. As a result, the TCBs can bind to both the T-cells 24 and antigen 23′ thus mediating T-cell activation and epithelial cell killing. Therefore, scenario C′ mimics a patient with a healthy gut receiving TCBs via systemic administration. A healthy gut represents a gut with a tight epithelial barrier, which is thus essentially impenetrable for TCBs and T-cells. In scenario C-1 and C-2 the antigen 23 is located on the apical side of the epithelial cells. In scenario C-1, T-cells 24 and TCBs 25 are administered through the basal channel of intestine chip 2. As a result of the intact tight epithelial barrier, neither the T-cells nor the TCBs can bind to antigen 23. Thus, no TCB mediated T-cell activation and epithelial cell killing is observed. In contrast, in scenario C-2, T-cells 24 and TCBs 25 are administered to the apical channel. As a result, the TCBs bind to both, the T-cells and antigen 23, mediating T-cell activation and epithelial cell killing. Scenario C-2 therefore mimics a pathologically leaky gut, for example that of a clinically plausible cancer patient condition of luminal TCB and T-cell penetration. Typically, immune cell recruitment to the epithelial cell can be visualized by known spectroscopic methods, such as confocal microscopy or Raman spectroscopy. Activation of the immune cells is typically determined by detecting specific cytokine release. A further parameter, which is measured in step S3 is the permeability of the intestine barrier after exposure to TCBs and T-cells. For example, the permeability is measured after a certain time interval, such as 12 h, 24 h or 48 h. A suitable method known in the art for measuring the integrity of the intestine membrane is transepithelilal/transendothelial electrical resistance (TEER), and the use of fluorescent marker (such as FITC-Dextran).

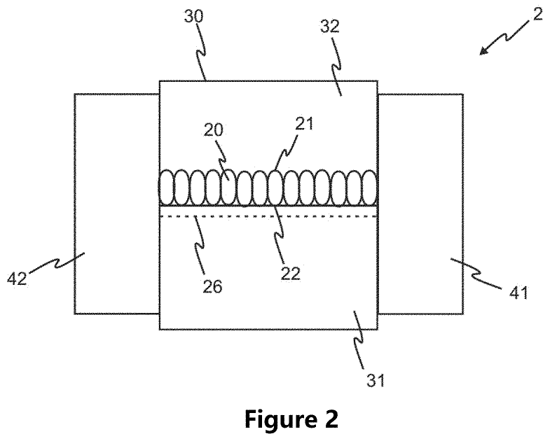

FIG. 2 shows an exemplary embodiment of an intestine chip 2 as used in the method according to the invention. Intestine chip 2 comprises main channel 30, which is divided into basal channel 31 and apical channel 32 by epithelial cells 20 and membrane 26. Furthermore, main channel 30 is flanked by two vacuum channels 41 and 42, which mimic the peristaltic movement of the gut.

The workflow described in FIG. 1 was applied in a model study, using TCBs recognizing the tumor-overexpressing proteins STEAP1 (six-transmembrane epithelial antigen of the prostate) and CEA (carcinoembryonic antigen). The TCBs comprised a CD3 arm binding to the CD3 receptor of on the T-cells to be activated and a target arm binding to the target antigen on the surface of the intestinal epithelial cells. Control antibodies comprise similar arms but lacking the ability to either bind to CD3 (herein referred to as non-CD3 antibody) or to the target (herein referred to as non-targeting CD3 antibody). STEAP1 and CEA are also expressed in healthy intestinal epithelial cells on the basolateral and apical surfaces respectively. Caco-2 colonocytes express STEAP1 and CEA and can form a polarized monolayer with apical and basolateral sides when cultivated in the intestine chip, thereby providing a relevant in vitro model for evaluating STEAP1 and CEA TCB intestinal liabilities.

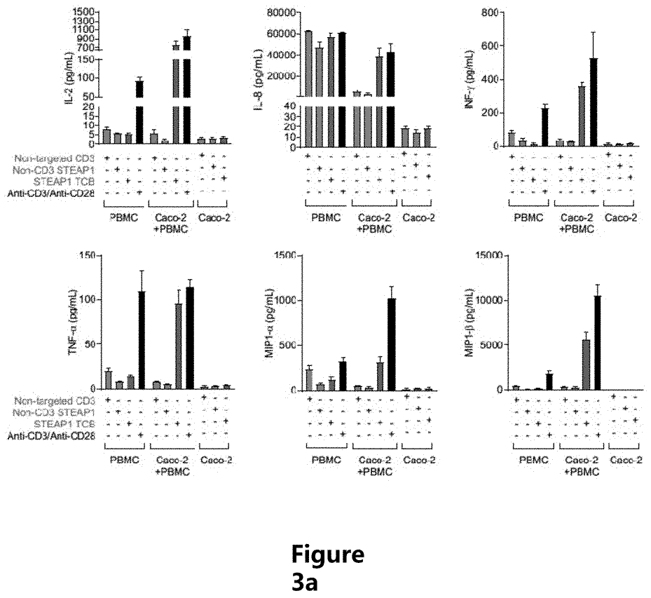

Prior to profiling the TCBs in the intestine chip, their potency and specificity in conventional Caco-2 monolayer culture in the presence of PBMCs was verified. It has been found that incubation of STEAP1 TCB with Caco-2 cells and PBMCs led to a substantial release of IL-12, INF-γ- [symbol] , MIP-1α- [symbol] , MIP-1β- [symbol] and TNFα- [symbol] , compared with TCB incubated with Caco-2 cells or PBMCs alone (FIG. 3a). Therefore, and as expected, TCB-mediated activation occurred in a target-dependent manner, requiring the presence of both target (epithelial) and effector (immune) cells. The control antibodies appeared inert (non-CD3 STEAP1) or much less potent (non-targeted CD3) than STEAP1 TCB, further underlining the target specificity of TCBs. As a positive control of T-cell activation, PBMCs co-treated with anti-CD3 and anti-CD28 antibodies triggered activation of PBMCs in the absence of target.

Surprisingly however, CEA TCB administered to the CEA-expressing Caco-2 cells (FIG. 3b) did not induce a cytokine release. Potential factors that could lead to the observed lack of activation include low potency of the TCB molecule and low CEA expression at the cell surface. To distinguish between these possibilities, the experiment was repeated using the CEA-high SNU-1544 cancer cell line as a positive cell control. CEA TCB treatment in the presence of SNU-1544 cells resulted in substantial cytokine release, confirming the potency of the TCB, and suggesting that the lack of activation in the presence of Caco-2 monolayers may be attributed to low or delocalized CEA expression by the latter. Noticeably, the non-targeted CD3 control antibody led to a substantial cytokine release, including in the PBMC-only conditions and at a higher extent than observed in FIG. 3a. These data suggest a CD3-mediated, target-independent T cell activation that can vary from PBMC donor to donor.

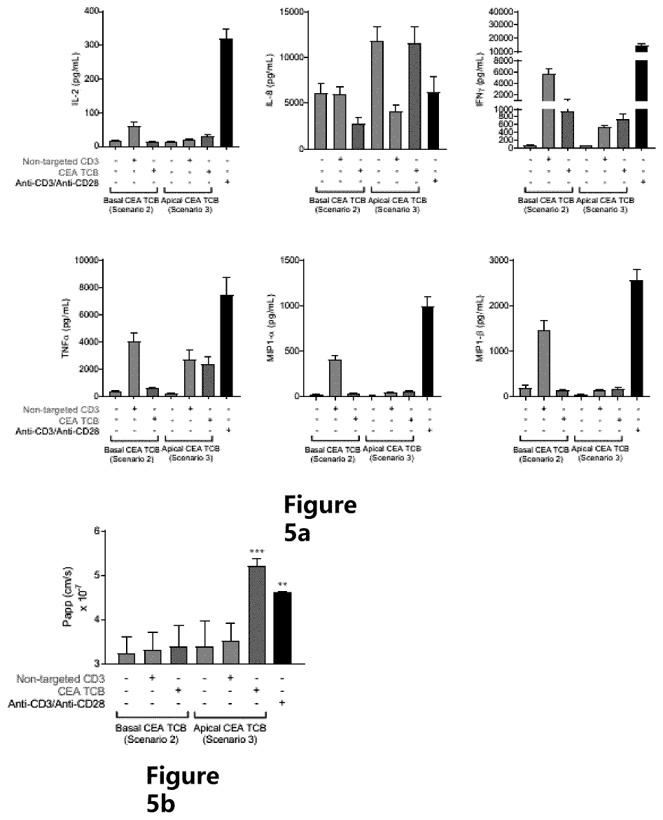

Intestine chips have been reported to promote the polarization and maturation of Caco-2 barriers, and the establishment of in vivo like morphology, by incorporating key environmental parameters, such as luminal flow and peristalsis, which are absent in conventional monolayer culture. It is hypothesized that intestinal barriers formed within the intestine chip may mimic the native intestinal epithelium with higher fidelity. In particular, it is postulated that CEA expression within the intestine chip may reflect the in vivo pattern more closely. Thus, the localization and expression of CEA in both the 2D monolayer cell culture and the intestine chip has been determined. Indeed, immunofluorescence analysis and confocal microscopy revealed poorly polarized expression of CEA within conventional Caco-2 monolayers, with a peak of CEA expression located more basally than ZO-1 (Zonula occludens-1) (FIG. 6a). In contrast, strong apical enrichment of CEA was detected within the intestine chip (FIG. 6b), verifying the above mentioned hypothesis. The chip likewise recapitulated the physiological expression of STEAP1 which was enriched in the basolateral regions of the epithelium (FIG. 6c).

After confirming the expression and polarization of CEA and STEAP1 on chip, TCB delivery to an intestine chip rendered immune-competent by the continuous circulation of PBMCs was performed next. Bearing in mind the mode of action of TCBs, which relies upon direct contact between antibody, effector and target cells, it was sought to determine whether PBMCs introduced through the basal channel were able to infiltrate the ECM layer and transmigrate to the epithelial barrier. Monitoring the presence of CFSE-labelled (carboxyfluorescein succinimidyl ester) PBMCs, extensive attachment of immune cells to the bottom of the membrane was observed, along with a small number of cells embedded in the epithelium. Thus, the aptitude of PBMCs to infiltrate the barrier, which is a key step for accessing the target site and mediate the TCB response on epithelial tissue was confirmed.

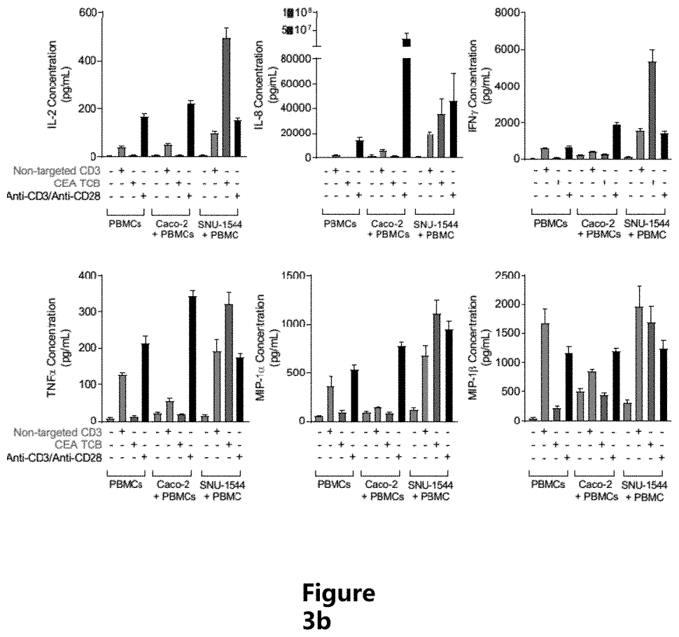

To assess whether a systemically administered TCB can engage at a basolaterally-expressed target protein and induce a T cell activation (scenario C′ in FIG. 1), STEAP1 TCB and PBMCs were introduced in the basal channel of the intestine chip and cytokine release in the supernatant was measured using multicomponent ELISA (FIG. 4a). As expected, basally delivered STEAP1 TCB resulted in substantial cytokine release (FIG. 4a). As reported for similar experiments performed in 2D (FIG. 3), the non-targeted CD3 control antibody elicited a cytokine release response, suggesting a CD3-driven, target-independent T-cell activation. Nevertheless, the potency of STEAP1 TCB was significantly superior at similar concentration (100 μg/ml). To assess TCB-mediated tissue damage, epithelial barrier permeability was measured 48 h after the introduction of STEAP1 TCBs and PBMCs. Substantial alterations in barrier function in response to STEAP TCB treatment were detected (FIG. 4b). Epithelial effects appeared to be target-dependent: significant changes in permeability were induced by TCBs in a concentration-dependent manner, and the control antibodies preserved the barrier integrity (FIG. 4b).

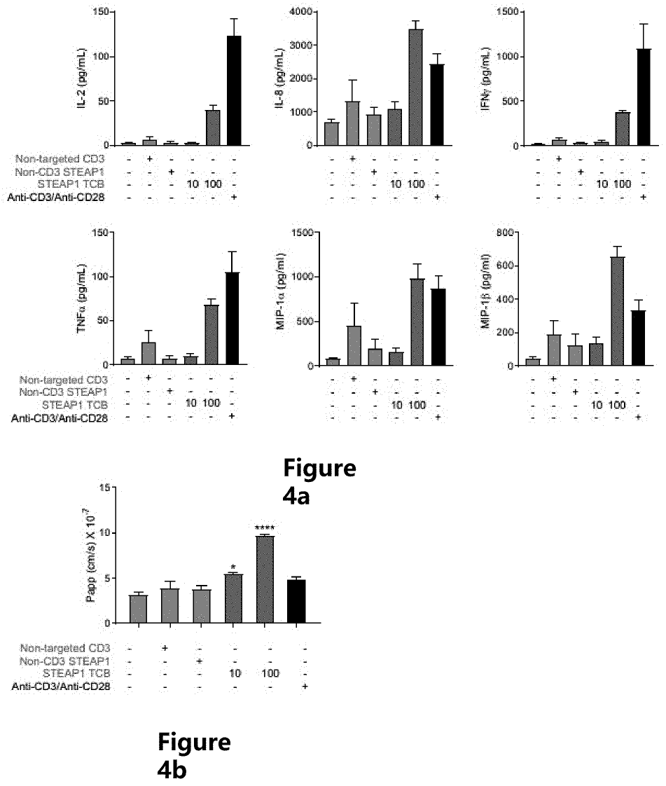

Next, the effect of CEA TCB, which recognizes an apically-expressed target, was evaluated in healthy versus leaky conditions by introducing CEA TCB and PBMCs in the basal channel (scenario C-1 in FIG. 1) or apical channel (scenario C-2 in FIG. 1) of the intestine chip respectively. As expected, CEA TCB administered with PBMCs on the luminal side induced a cytokine release response, best exemplified by TNFα concentration in the supernatant (FIG. 5a). However, in this experiment the overall cytokine profile appeared highly variable and unspecific: The non-targeted CD3 control antibody and the CEA TCB in the basal conditions triggered a strong cytokine release response, hypothetically resulting from a donor-specific pre-conditioned state of PBMCs that may confound the target-dependent potency of CEA TCB. Such poised state might be exacerbated by the high T cell density and elevated anti-CD3 drug concentrations used. Nevertheless, and in accordance with STEAP1 TCB data, the effect of CEA TCB on epithelial tissue damage appeared to be target dependent: Significant changes in permeability were induced by CEA TCB in an accessibility-dependent manner (scenario C-1 vs C-2), and the control antibodies preserved the barrier integrity (FIG. 5b). Thus, TCB-mediated cytokine release and tissue damage appeared to be uncoupled, at least partially, under in vitro conditions, and measurement of barrier integrity appears to be a more accurate indicator of target-dependent TCB-mediated effects.

Overall, the data presented herein have important implications for the predictability and clinical impact of organ-on-a-chip models for drug profiling: Despite CEA expression in Caco-2 cells culture in 2D, CEA TCB failed to elicit immune cell activation (FIG. 3b), whereas CEA TCB treatment of Caco-2 barriers within the intestine chip resulted in activation and barrier leakiness (FIG. 5b). The differential outcomes can likely be attributed to the higher extent of epithelial maturation and physiological pattern of CEA expression observed within the chips, compared with conventional Caco-2 monolayers. This finding highlights a crucial difference between the intestine chip and conventional approaches for the target tissue-specific profiling of TCBs, with profound clinical implications. An assessment of TCB activity based on standard approaches would deem the CEA TCB ‘safe’ for off-tumor, on-target intestinal effects—a predication with potentially dire consequences in the clinic. The intestine chip, in contrast, would predict low intestinal liability only in patients with intact intestinal barriers, whereas potential on-target TCB effects are foreseen in the clinically plausible and frequent case of patients with leaky guts.

The application of the immune-competent intestine chip has thus been demonstrated as a platform for TCB profiling in polarized epithelia such as the gut. In particular, the method was able to capture TCB-mediated target tissue damage specifically related to a target antigen. This demonstration was directly enabled by the modularity of the system, which enabled independent access to the luminal and basal epithelial surfaces, thereby allowing to distinguish target-dependent from non-specific, off-target effects, which would otherwise be unapparent in conventional monolayer culture. Moreover, the physiologically-relevant micro-environmental parameters, i.e. presence of ECM and flow, afforded by the intestinal chip promoted the maturation and polarization of the intestinal barrier. The physiologically faithful antigen presentation by intestinal epithelium formed within the intestine chip proved to be essential for revealing TCB effects that were unforeseen using poorly polarized intestinal cells in 2D culture. Whereas TCB safety profiling in the gut was chosen to showcase the utility of this platform, the paradigms introduced here are readily adaptable and extendable to conducting both safety and pharmacology studies of standard antibody or small molecule effects.

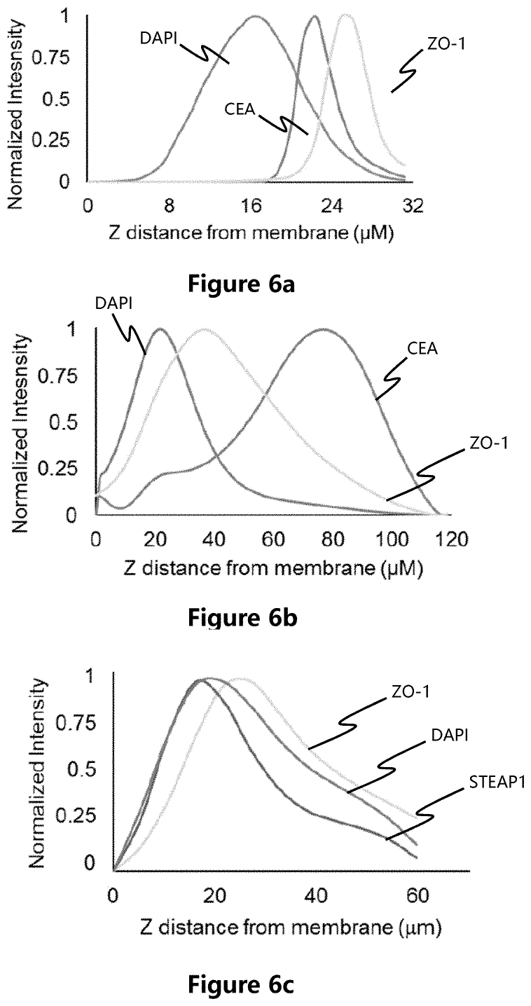

In addition to the intestine chip containing Caco-2 cells, a further embodiment of the invention uses an intestine chip comprising primary epithelial organoids. These were derived from the healthy region of a colonic biopsy and were expanded using standard Matrigel-based culture and subsequently plated on the intestine chip, where they form a tight, polarized and differentiated intestinal epithelial monolayer. The basal channel of the corresponding chip comprises an endothelial barrier, formed from human intestinal microvascular endothelial cells (HIMECs). In order to determine whether the findings relating to CEA TCB with Caco-2 cells can be recapitulated with the intestine chip comprising primary epithelial organoids, expression and the apical localization of CEA was confirmed by immunofluorescence analysis. Additionally, the luminal channel of the device was used to co-deliver PBMCs and CEA TCB, or a control, non-targeting CD3-only engaging TCB (DP47). CEA TCB treatment led to a significantly increased PBMC attachment to the primary epithelium, which is consistent with the TCB mode of action (FIG. 7a). With respect to barrier permeability, a CEA TCB induced dose-dependent increase in barrier permeability is observed, as illustrated in FIG. 7b. This suggests that TCB treatment entail epithelial cell killing and barrier damage. In conclusion, the CEA TCB-related findings were replicated with the intestine chip with primary epithelial organoids, making it a highly physiologically relevant option for the safety profiling if TCBs.

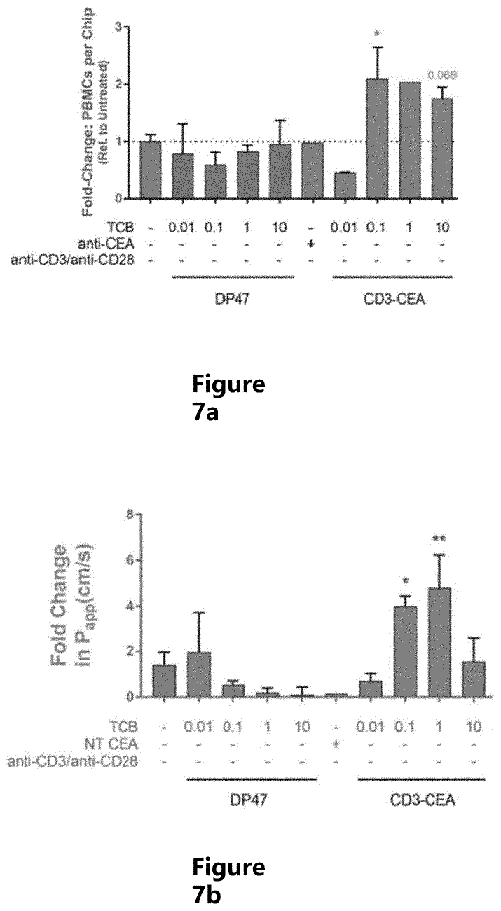

FIG. 8 further demonstrates that the method disclosed in any of the herein described embodiments is also suitable for comparing and differentiating the safety effects of a related compounds, and thus allows for assessing the safety of a new therapy at a very early stage. In the example shown, two CEA-targeting TCBs were assessed, namely the above mentioned CEA TCB and CEACAM5 TCB. CEACAM5 TCB is a higher affinity molecule, thus resulting in more pronounced intestinal toxicity in patients, which primarily manifests as severe diarrhea. As already discussed in the context of the assessment of CEA TCB (see above), the apical delivery was performed, in order to ensure target engagement. TCB mediated effects were assessed by considering cytokine release and T cell activation (CD69 expression) at 72 h, and through live monitoring of epithelial cell apoptosis (caspase activation) at 24, 48 and 72 h after TCB/PBMC treatment. As can be seen from FIG. 8A, CEACAM5 TCB induced robust, dose dependent cytokine release at relevant doses. FIG. 8B further demonstrates a significantly enhanced T cell activation by CEACAM5 TCB. Additionally, an increase in epithelial cell apoptosis was observed (FIG. 8C). By comparison, CEA TCB resulted in substantially lower cytokine release and T cell activation, and no detectable changes in apoptosis, consistent with its lower binding affinity and more favorable clinical safety profile. These data suggest that the intestine chip is capable of capturing in vitro the different potencies and clinical safety effects of TCB molecules, which further qualifies it as a suitable platform for the safety assessment of new therapies.

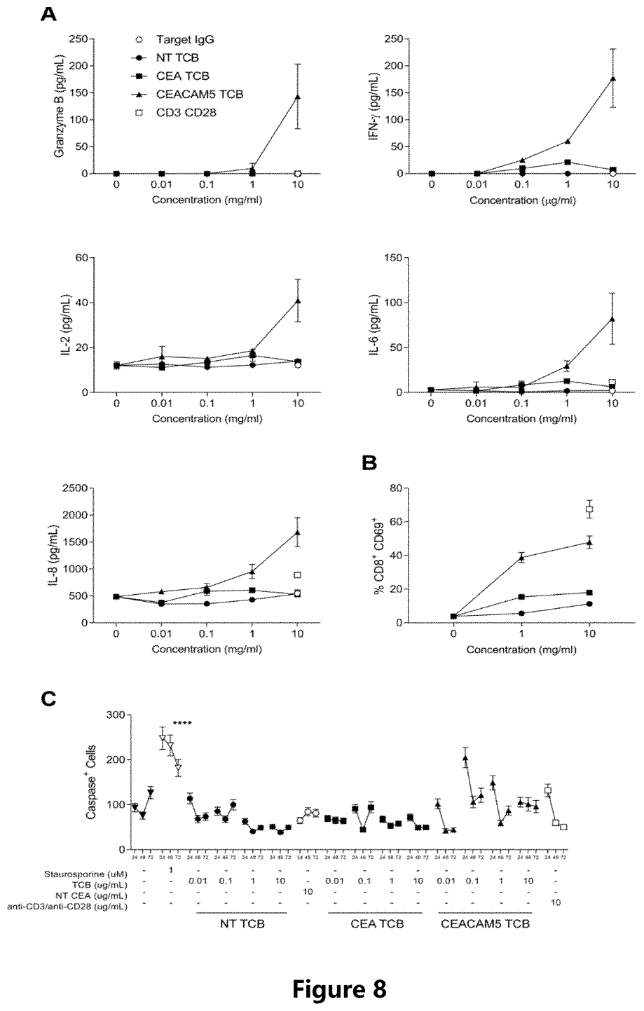

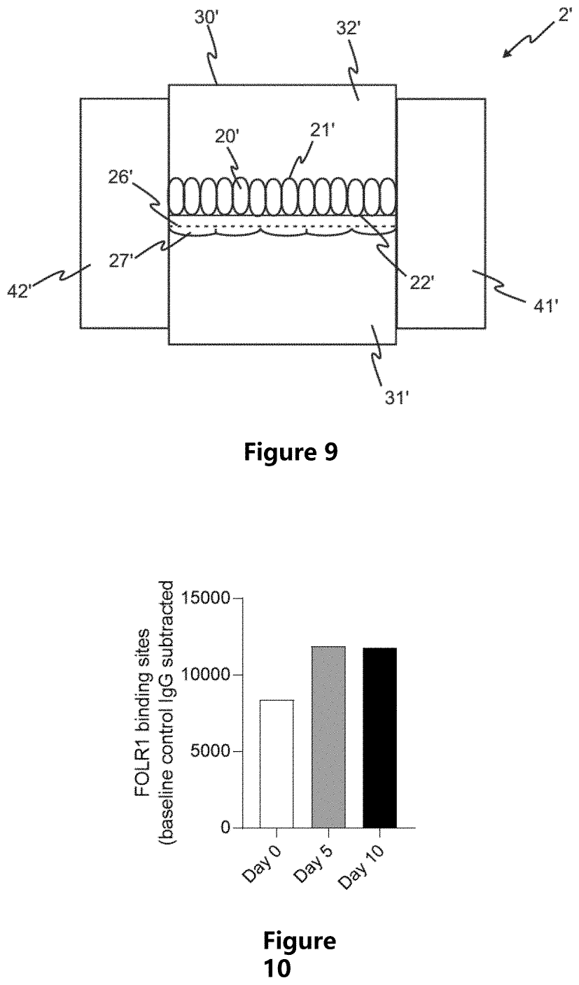

FIG. 9 shows an exemplary embodiment of a lung chip 2′ as used in the method according to the invention. Lung chip 2′ comprises main channel 30′, which is divided into basal channel 31′ and apical channel 32′ by epithelial cells 20′, pulmonary microvascular endothelial cells 27′ and membrane 26′. Furthermore, main channel 30′ is flanked by two vacuum channels 41′ and 42′, which mimic the breathing movement of the lung. Prior to testing FOLR1-TCBs, expression of FOLR1 in chips was verified by immunofluorescence analysis and surface expression quantified by flow cytometry (FIG. 10). Following the verification of physiological target expression, TCBs and T-cells are introduced into the basal channel or the apical channel. Furthermore, a parameter of the administration is measured, in particular TCB-mediated pharmacological parameters and safety parameters are measured. If the target and T cells are present in the same channel, the TCBs bind to both, the T-cells and antigen, mediating T-cell activation, attachment and epithelial cell killing. That scenario is equivalent to the Scenario C-2 of the gut chip (FIG. 1) and therefore mimics a pathologically leaky alveolus, for example that of a clinically plausible cancer patient condition of luminal TCB and T-cell penetration. Typically, immune cell recruitment to the epithelial cell can be visualized by confocal microscopy. Activation of the immune cells is determined by detecting specific cytokine release and measuring surface expression of activation markers by flow cytometry.

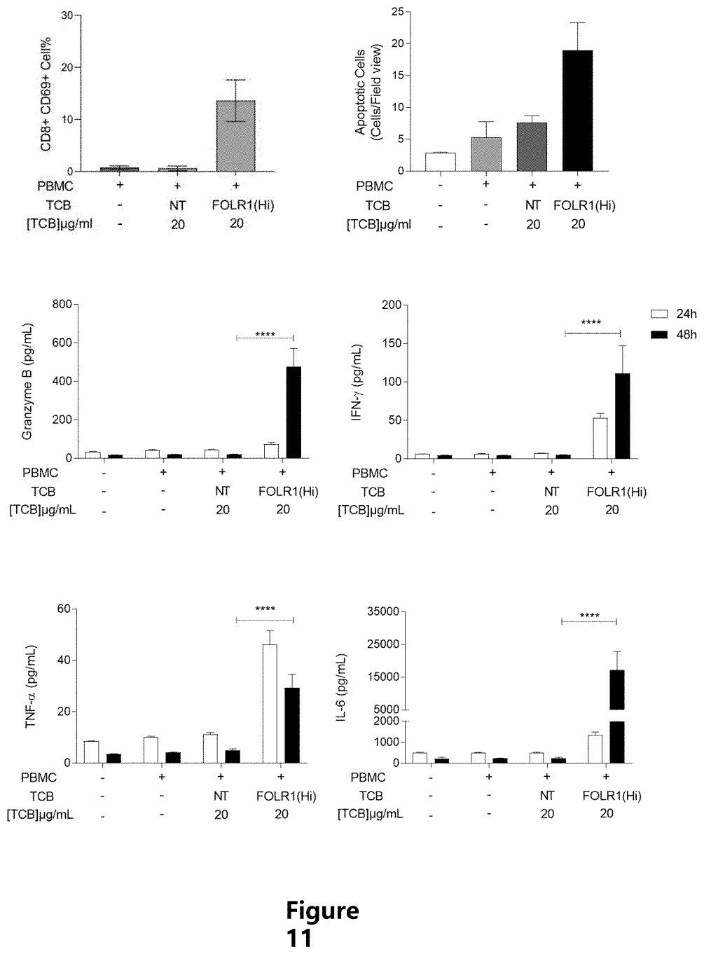

FIG. 11 shows TCB-mediated cytotoxicity and cytokine release in one embodiment of a lung chip, treated with 20 μg/ml of FOLR1 high affinity antibody (FOLR1(Hi) TCB). For performing the assay, the luminal channel was used to co-deliver PBMCs and FOLR1-TCB, or a control, non-targeting CD3-only engaging TCB (NT). Apoptosis detection by caspase 3/7 live imaging revealed a T-cell-dependent killing of alveolar epithelial cells upon FOLR1 TCB treatment. FOLR1-TCB also led to a significantly increased PBMC attachment to the primary alveolar epithelium, in accordance with the expected increased adhesion molecules due to T cell activation. The latter was quantified by flow cytometry detection of activation markers, which demonstrated the specific induction of T cell engagement in presence of FOLR1-TCB. Culture supernatants sampled at different time points were used for multiplex cytokines analysis and showed cytokines release induced by FOLR1-TCB treatment.

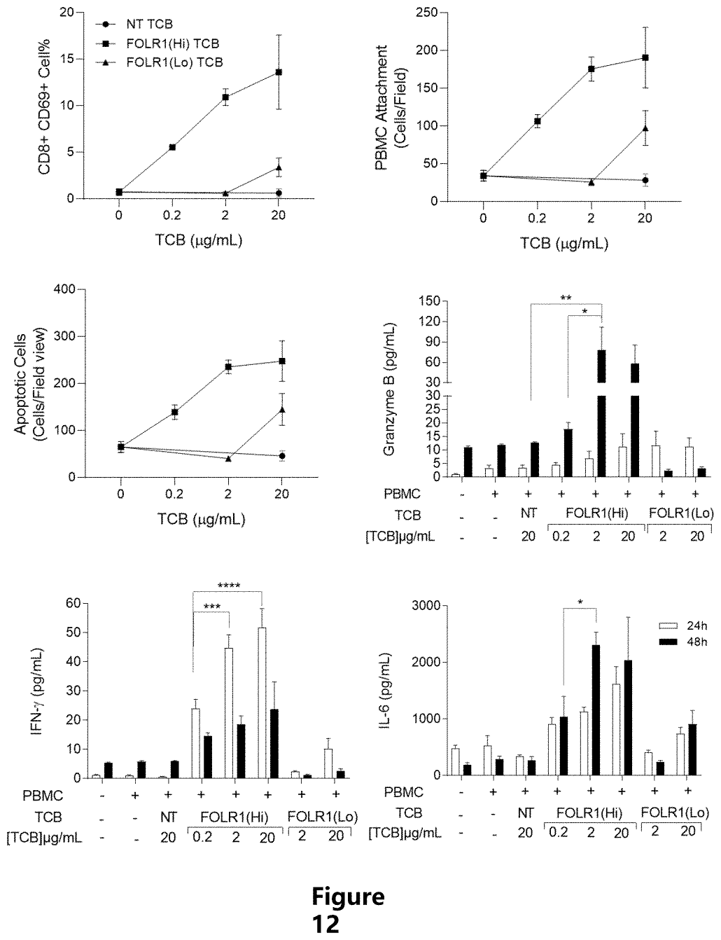

In another assay shown in FIG. 12, the lung chip was used to obtain pharmacological data and to compare to related TCBs with varying affinity. This embodiment of the lung chip captures antibody format differences demonstrating FOLR1(Hi) TCB (high affinity) has a greater lung epithelial toxicity than FOLR1(Lo) TCB (Low affinity). Graphs represent T-Cell activation (top left graph); T-Cell (PBMC) attachment to the Alveolus Epithelium (top right graph, after 48 h of treatment); apoptotic cell quantification, lower left and right graphs, respectively. Middle right and lower panels demonstrate a selective increase in cytokine secretion between FOLR1(Hi) TCB over FOLR1(Lo) TCB at around 2 μg/ml up to 20 μg/ml. Thus, the measured pharmacological parameters allow a comparison of two related TCBs, thereby demonstrating a safer profile for the low affinity FOLR1-TCP than for the high affinity FOLR1-TCB for apoptosis, PBMC attachment, T cell activation and cytokines release, which corroborats the findings of preclinical cynomolgus toxicity studies. This indeed proves that the lung chip in coculture with PBMCs constitutes a human relevant in-vitro alternative for safety profiling of TCBs.

Methods

Intestine Chip

Chip Activation and Deposition of Collagen I Gel on the Intestine Chip

The PDMS surface of the chip was activated as follows: the ER-1 reagent was resuspended in the ER-2 solution (Emulate, Inc) at a final concentration of 0.5 mg/ml and administered to fill the top and bottom channels of the chip. The ER-1 & 2 solution was activated for 20 minutes by UV treatment followed by sequential washes with ER-2 and PBS (ThermoFisher #14190144). An extracellular matrix (ECM) solution of 1 mg/ml rat tail collagen I (Corning #354249) to coat the top channel was prepared on ice according to the method of Doyle et al. 2017. Briefly, 10 uL of reconstitution buffer and 10 uL 10×DMEM (MilliporeSigma #D7777-10×1L) and collagen I were diluted in PBS supplemented with calcium and magnesium (PBS++) (ThermoFisher #14040133) and the pH adjusted with 1N Sodium hydroxide (MilliporeSigma #221465) to 7-7.5 for a final volume of 1 ml. A second collagen I ECM solution of 100 μg/ml to coat the bottom channel was prepared by diluting the 1 mg/ml solution 10-fold in PBS++. The remaining PBS in the top and bottom channels of the chip was then aspirated and the 1 mg/ml solution was added to the top while the 0.1 mg/ml solution was added to the bottom channel. The ECM solution in the top channel was allowed to polymerize at room temperature for 45 minutes followed by gentle perfusion of 200 μl PBS++ with a P200 Gilson Pipet until the semi-polymerized gel began ejecting from the chip. Perfusion was then stopped and the pipet tip was ejected to allow the remaining volume to perfuse by gravity flow. Collagen I gel was allowed to continue setting in a humidified 37° C. incubator for 1.5 hours. The unpolymerized collagen I was then removed from the top and bottom channels by perfusion with PBS++ and stored at 4° C. in a humidified petri dish for up to a day.

Seeding of the Caco-2 Intestine Chip

Caco-2 BBE epithelial cells for seeding the intestine chip were routinely subcultured as per vendor recommendations (Millipore Sigma 86010202). For seeding, Caco-2 cells were dissociated with 0.05% trypsin-EDTA (ThermoFisher 25300054) for 5 minutes in a 37° C. humidified incubator and resuspended with excess DMEM (ThermoFisher #10569044) supplemented with 10% fetal bovine serum (FBS) (ThermoFisher #16000) and 100 U penicillin streptomycin (pen/strep) (ThermoFisher 15140122). Cell density and viability was assessed by trypan blue (MilliporeSigma T8154-100ML) exclusion assay followed by centrifugation and resuspendion in DMEM media supplemented with 10% FBS and pen/strep to a final cell density of 1.5 million cells per ml. S1 Organ-Chips (Emulate, Inc.) were then seeded with Caco-2 and incubated for at least 1.5 hours at 37° C. to allow for cell attachment. Both top and bottom channels of the chip were then washed with 200 uL of media to remove unattached or dead cells.

Media Equilibration

Before connecting chips to Pods, bottom media, DMEM supplemented with 10% FBS and 100 U pen/strep and top media, bottom media supplemented with 20 μg/ml of 3 kDA dextran-cascade blue (ThermoFisher D7132), were equilibrated as follows to remove excess gas from medium and minimize potential bubble formation. 50 mls of top media was warmed in a 37° C. bath for 1 hour prior to being filtered through a 0.45 μm Steriflip (FisherSci #SE1M003M00) for at least 15 minutes with a vacuum source of −80 kPa. The equilibrated top media was then used immediately or incubated at 37° C. before use.

Priming Pods

Prior to connecting Chips with Caco-2 cells to Pods, the Pods were primed for liquid flow. 1-3 mls of top and bottom media were added to their respective channels reservoirs of the Pod, respectively. 300 μl of basal media was then added to both outlet reservoirs to cover the outlet “Vias”. The Pod ID was set to “054” and the “Prime” sequence of the Zoe Culture Module (Zoes) was then selected and run.

Connecting Chips to Pods and Zoes

Once primed, the Chips with Caco-2 epithelium were connected to the Pods, placed in a Zoe tray, and the tray inserted into the Zoe. The flow rate was set to 30 μl/hr and the “Regulate” cycle was run to initiate flow. Chips were checked for lack of bubbles and flow consistency monitored daily for the next 24-72 hours. If bubbles or inconsistent flow were detected, chips were first manually reprimed by pushing 1 ml of media through the input via and watching for bubble ejection and secondly, the “Regulate” cycle was re-run.

Caco-2 Culture on-Chip

The maturation of the Caco-2 epithelium was monitored daily by brightfield microscopy for the first 72 hours to assess the formation of epithelial villi-like structures following this initial monitoring period. Barrier function was routinely assessed by collecting inlet and outlet samples and measuring the apparent permeability of the dextran-cascade blue 3 kDa. The Caco-2 Intestine-Chip was considered mature between 7-14 days upon formation of a villi-like structures and an apparent permeability between 1×10-7 and 1×10-6 cm/s.

PBMC Isolation

PBMCs were isolated from citrated, fresh whole blood (Research Blood Components) using Lymphoprep (Stemcell Technologies Catalog #07851) reagent and following the manufactures recommended protocol and cryopreserved in 50% FBS, 10% DMSO (Millipore Sigma D2650-100ML), 40% RPMI-1640 (ThermoFisher #61870-010). A total of million PBMCs per cryotube were frozen in a Mr. Frosty Container (VWR #55710-200).

2D TCB Plate Validation Experiment

100K Caco-2 Cells were plated in each well of a 24-well plate. Cells were cultured for 2-4 days until a fully confluent monolayer of cells was observed. The day of the experiment, cryopreserved PBMCs were thawed and resuspended in DMEM supplemented with 10% FBS and 100 U of pen/strep. Cells were then labelled with 5 μm CSFE (ThermoFisher #C34554) for 20 minutes at room temperature. Excess CSFE was then removed by pelleting cells and washing twice with media. Labelled PBMCs were then resuspended to a final density of 1 million cells per ml. The media in each treatment well was then aspirated and replaced with 0.5 ml of the PBMC solution. Treatment antibodies were then added and the plates incubated overnight at 37° C. The following day, the media from each well was triturated and collected in V-bottom 96-well plates (VWR #29442-068). The PBMCs were pelleted by centrifugation at 300 g×5 min and the supernatant transferred to a second 96-well plate for measurement of cytokine levels by multi-analyte ELISA (Meso Scale Discovery #K15067L-1) or viability by LDH (Promega #G1780). Leftover supernatant was decanted from the pelleted PBMCs and fixed with the BD cytofix solution (ThermoFisher #BDB554714). After fixation, PBMCs were spun down at 300 g for 5 minutes and the fixation solution decanted. PBMCs were either stained immediately for analysis by flow cytometry (FACs) or resuspended in 95% FBS, 5% DMSO and stored at −80° C. The remaining Caco-2 wells were then either fixed with 4% paraformaldehyde (PFA) (FisherSci #50-259-98) for 15 minutes at room temperature or lysed for measurement of intracellular LDH according to the manufacturer's recommendations. PFA was decanted from cells, replaced with PBS and stored at 4° C. until staining for immunofluorescence. Fixed Caco-2 wells were prepared for IF staining by first blocking with cell staining solution (Biolegend #420201) for 30 minutes at room temperature. Anti-human CD69 (ab201570-100UG) was diluted 100-fold in cell staining solution and incubated overnight at 4° C. Wells were then washed 3-times with 0.5 ml PBS with 5 min rest periods in between each wash. CD69 was then labelled with donkey anti-mouse conjugated to Alexa 647 secondary antibody (ThermoFisher A-31571) and diluted 250-fold in cell staining solution. Excess stain was washed three times again with PBS and a 5 min rest in between each wash. Cells were then stained with a 100-fold dilution of anti-human ZO-1 conjugated to Alexa 555 (ThermoFisher #MA339100A555) in cell staining solution overnight at 4° C. Excess stain was washed away 3-times with PBS with a 5 min rest in between steps. Nuclei were then stained with 4 drops per ml of NucBlue (ThermoFisher #R37605) in PBS or a 2,000-fold dilution of Hoescht (ThermoFisher H3570). Stained cells were imaged immediately or protected from light and stored at 4° C.

Administration of PBMCs to Caco-2 Intestine-Chip

Previously cryostored PBMCs were thawed in a 37° C. bead bath and resuspended in DMEM supplemented with 10% FBS and 100 U of pen/strep to a final concentration of 2 million cells per ml and labelled with a final concentration of 5 μM CSFE as described above. After removing excess CSFE, PBMCs resuspended to a density of 16.7 million cells per ml in basal media, aliquoted to 6-well plates, and antibody treatments were added. The final density was 8.3 million PBMCs per ml and the incubation lasted for 4 hours. Aliquots of PBMCs in the treatment conditions were collected, fixed for later FACS analysis, and the supernatants frozen at −20° C. for measurement of cytokine levels. During the PBMC incubation on plates, samples from chips were collected to assess barrier function, and brightfield images were collected to assess epithelial morphology. After 4 hours of incubation, PBMCs to be basally administered were diluted to 50% Percol (17-0891-02) (v/v) for a final density of 4.17 million PBMCs per ml. For PBMCs to be apically administered, apical media supplemented with 40 μg/ml dextran 3 kDa-cascade blue was added for a final density of 4.17 million PBMCs per ml. The respective inlet channels were aspirated from each treatment condition and 1.5 ml of PBMC suspension was added to the inlet reservoirs. 200 μl of the PBMC treatment conditions were then added to a 96-well plate in triplicate for a side-by-side plate comparison. The chips were equilibrated rapidly with PBMCs by flowing at 300 μl/hr for 30 minutes. Both outlet channels were then aspirated and flow was restarted. The flow rate was set to 30 μl/hr for the top and bottom channels except for chips with basally administered PBMCs with 50% Percol whose bottom channel flow rate was set to 60 μl/hr to account for the increased viscosity. This was considered timepoint 0 for the experiment. Flow was interrupted after 6 and 24 hours and inlet and outlet samples collected for readout analysis on V-bottom plates. For apically administered PBMCs, the inlet PBMCs reservoirs were triturated to resuspend the PBMCs and flow restarted. For cytokine and FACs analysis, 25 μl of collected samples were transferred to V-bottom plates and diluted at least 5-fold with PBS. PBMCs were pelleted by centrifugation at 300 g for 5 minutes and the supernatant transferred for cytokine analysis. The PBMC pellets were fixed and stored as described previously for FACs analysis. All cytokine and barrier function samples were sealed in parafilm and stored at −20° C. until ready for analysis.

After 24 hours of PBMC administration, samples for readouts were collected as described previously. Chips were then disconnected from Pods and fixed with 4% PFA for 15 minutes at room temperature, washed with 200 μl PBS for each channel, and cut in half, submerged in PBS supplemented with 0.05% sodium azide (VWR #101446-792), and at 40 C for storage.

Immunocytochemistry (ICC)

Chips were blocked with Cell Staining Solution for 0.5 hours at room temperature. Primary antibodies for anti-human antigens STEAP1 (Abcam ab207914), CEA (Abcam ab133633), CD45 (ThermoFisher MA517687), CD69 (Abcam ab201570), ZO-1 conjugated to Alexa-555 (ThermoFisher #MA339100A555) were diluted 100-fold in Cell Staining Solution and incubated on chips overnight at 4° C. Chips were then washed 3-times with PBS at room temperature including a 5-minute rest period between washes. Donkey anti-mouse Alexa-647 (ThermoFisher A-31571), anti-rat Alexa-555 (ThermoFisher SA510027), and anti-rabbit Alexa-647 (ThermoFisher A-31573) secondary antibodies were diluted 500-fold in Cell Staining Solution and incubated at room temperature for 1.5 hours and protected from light. Chips were then washed 3-times with PBS at room temperature with 5-minute rest periods between washes. Nuclei were counter stained with either NucBlue (ThermoFisher R37605) diluted 4 drops per ml of PBS or Hoechst (ThermoFisher H3570) diluted 2000-fold in PBS for 20 minutes at room temperature.

Confocal Imaging

Stained Chips were imaged on a LSM880 Confocal microscopy with a 20× objective with a NA=0.6 with Airyscan mode enabled. Z-stack image planes were collected with an optimal step size to match the Nyquist frequency of 1.5 μm per plane. For each antigen, 2 chip replicates and 3 fields of view (FOV) were imaged. Airyscan processing was performed with the Zen Blue software package.

Epifluorescent Imaging

The stained, half chips were imaged on an Olympus epifluorescent microscope (IX83 Research System) at 10× and 20× magnifications to produce tiled, Z-stacked images of the chip epithelium. Quantification of immune cell migration into the epithelial compartment was assessed by analyzing each plane of a 7-plane Z-stack, 15-tile tile collected at 20× magnification for CD45 signal. Immune cells were detected using the MATLAB “imfindcircles” function that locates circles using a Hough transform. Analysis was performed on 2-3 Chip replicates per experimental condition.

Assessment of Epithelial Antigen Polarization

Confocal Z-stacked images of Chip antigen staining were visualized by the Fiji software suite (Schindelin, J.; Arganda-Carreras, I. & Frise, E. et al. (2012)). The expression of target localization was determined by averaging the intensity values of each XY plane at each Z-position. The Z-profile of each color channel was normalized by the maximum and minimum average intensities according the following equation:

$$( \frac{{I}_{i} - {I}_{m i n}}{{I}_{m a x} - {I}_{m i n}} )$$

Antigen polarization was assessed by comparing the Z-plane of the average maximum intensity (Zₘₐₓ) to that of the apically polarized tight junction protein ZO-1:

ΔZₘₐₓ=(Zₘₐₓ^{antigen}−Zₘₐₓ^{ZO-1})

Differences in ΔZₘₐₓ≥0 indicated apical polarization while ΔZₘₐₓ<0 indicated basolateral polarization.

Measurement of Cytokine Levels

Supernatants collected from chips and plates were analyzed by Meso Scale Discovery multi-analyte kits (K15067L-1) according to manufacturer's instructions.

Flow-Cytometry (FACs)

PBMC suspensions stored at −80° C. were thawed and centrifuged at 300 g for 5 minutes at room temperature. Supernatants were decanted and V-bottom plates gently vortexed to loosen the cell pellets. Cell suspensions were then washed twice with 200 μl PBS. Antibody staining solutions were prepared by diluting anti-human CD69 (BioLegend 310910) conjugated to APC 250-fold in Cell Staining Solution and incubated 1.5 hours at room temperature or overnight at 4° C. Following antibody staining, cells were washed 3 times with PBS with a 5-minute rest period in between washes.

Labelled PBMCs were run on a BD FACs Canto II instrument at the Harvard Digestive Disease Center at Boston Children's Hospital and analyzed with the FlowJo V10 software suite.

Barrier Function

Barrier function was assessed by measuring the apparent permeability of dextran 3 kDa-cascade blue from the top to the bottom channel. Briefly, samples were collected from the top and bottom channel inlet and outlet reservoirs and stored at −20° C. until the day of measurement. An 8-point standard curve was prepared by 1-to-1 serially diluting the apical inlet media with basal inlet media. All samples and the standard curve were diluted 3-fold in PBS for a final well volume of 150 μl and the fluorescence intensity of the labelled dextran was measured on a BioTek Synergy Neo instrument. Autogain was performed on the standard curve to set the appropriate fluorescence dynamic range.

The apparent permeability of the labelled dextran was calculated using the equation:

$${P}_{app} = ( \frac{dQ / dt}{{C}_{0} \times A} )$$

Where C₀ is the input concentration of dextran measured in from the apical inlet channel tracer, A is the surface area of the Chip membrane, and dQ/dt is the flux of the dextran transiting from the top to the bottom channel. The flux was calculated by measuring the number of dextran molecules that had moved from one channel to the other.

Lung Chip

Chip Activation and Deposition of Collagen I Gel on the Intestine Chip The same procedure than the intestine chip was followed except that ECM of the top channel was constituted of 200 μg/mL Collagen IV, 30 μg/mL Fibronectin and Laminin 5 μg/mL, whereas the ECM of the bottom channel was constituted of 200 μg/mL Collagen IV and 30 μg/mL Fibronectin.

Seeding of the Caco-2 Intestine Chip Each channel was washed with 200 μL complete SAGM culture medium before seeding human alveolar epithelial cells at 1×106 cells/mL density, with 35 to 50 μL of the cell suspension into the top channel inlet port while aspirating the outflow fluid from the chip surface. Chips were then placed at 37° C. for 2 hours or until cells have attached. A gentle medium wash was performed to remove excess media and chips placed back in the incubator. On day 1 and 2, complete maintenance medium was replenished in top and bottom channel of each chips. Droplets of complete medium was also added to fully cover all inlet and outlet ports to prevent evaporation. On day 3, 200 μL of complete EGM-2MV culture medium was introduced to the bottom channel of each chip before seeding human microvascular endothelial cells at 5×106 cells/mL density, with 15 to 20 μL of the cell suspension into the bottom channel. A cradle was used to place the chips upside down while endothelial cells needed to attach for one hour at 37° C. A wash with 200 μL was then performed for all top and bottom channels with complete maintenance medium or EGM-2MV respectively. Chips were placed backed in the incubator until connection to Zoes.

Alveolar Epithelial Cells and Endothelial Cells Culture On-Chip

On day 4 of the culture, chips were connected to Zoes and on day 5 air-liquid-interface was introduced in the upper channel by using complete aspiration technique followed by a one-minute step of 1000 μL/hour upper channel flow rate and opt/hour bottom flow rate. Inlets and outlets reservoirs were then emptied and a microscope check performed before repetition of the previous flow step. Inlet and outlet reservoirs were emptied again, but this time leaving a small liquid layer over the bottom inlet reservoir to prevent introduction of unwanted bubbles during the flow. 2-4 mL complete Air Liquid Interface (ALI)-medium was then introduced to bottom channel inlet reservoirs and 1 mL of medium in the air channel Pod inlet reservoirs immediately followed by 1 mL addition in the outlet reservoirs. This equal media distribution in the Pod reservoirs is required to maintain static pressure in the air channel. All trays were then placed back to the Zoes and top channel set to “Air” while bottom channel flow rate set to 30 μL/hour. Medium was refreshed in the bottom channel inlet reservoirs every other day or as needed.

Administration of PBMCs and TCBs to Lung Chip

One day prior to PBMC addition, medium from inlet and outlet reservoirs was aspirated and replaced by hydrocortisone-free ALI medium. A bubble check was performed and ALI maintained until the day after. Cryopreserved PBMCs were thawed and kept at 2)(106 cells/mL in complete medium overnight. On dosing day, PBMCs were harvested, counted, washed and stained for 20 minutes at 37° C. with 5 μM CMFDA Cell Tracker Green (or equivalent dye) according to manufacturer's instructions. PBMCs were then washed and resuspended in PBMC dosing media (M199+2% FBS) at a concentration of 8×106 cells/mL. 2×TCB-media solution was added to the cells and incubated for 1 h at 37° C. 500 μL of ALI-top channel media (M199+2% FBS) were added to the top inlet reservoirs of all chips before running a flush cycle to re-introduce liquid-liquid interface (LLI). Top channel flow rate was set to woo 4/hour and bottom to 0 and allowed to run for 3 minutes. Zoë were paused and all media aspirated from top channel outlet reservoir. 500 μL of the dosing solution were then added to the top channel inlets in appropriate chips, a flush cycle run at explained previously before checking PBMCs distribution under the microscope. Chips were connected back to Zoe and the top channel left static for 2-3 hours to allow the PBMCs to settle down. After that time, top channel inlets and outlets were emptied and refreshed with ALI-top channel media. The bottom channel inlets and outlets were also emptied and filled with fresh ALI media (without hydrocortisone). Chips were then connected to flow rate of 30 μL/hour for both channels.

QIFIKIT

Epithelial cells were detached from the chips with TrypLE and washed before determining cell surface antigens with QIFIKIT with anti-FOLR1 primary mouse monoclonal antibody (R&D systems, Catalog #MAB5646) following manufacturer's instructions (K0078-QIFIKIT, Dako).

Measurement of Cytokine Levels

For Luminex assays, cell-free tissue culture supernatants were collected 24 and 48 h post-treatment and analyzed for cytokine/chemokine levels using Custom ProcartaPlex Human 13-plex Cytokine & Chemokine Panel from Thermo Fischer Scientific according to manufacturer's instructions.

1. A method for assessing a compound interacting with a target on polarized epithelial cells, the method comprising:
providing an organ chip comprising a main channel and polarized epithelial cells, wherein the main channel is divided into an apical channel and a basal channel separated by the polarized epithelial cells, wherein the apical side of the polarized epithelial cells is directed towards the apical channel and the basolateral side of the polarized epithelial cells is directed towards the basal channel;
determining the localization and optionally the expression level of the target on the polarized epithelial cells;
administering the compound and optionally immune cells, such as peripheral blood mononuclear cells (PBMC) to the basal channel, when the target is localized on the basolateral side of the epithelial cells, or to the apical channel, when the target is localized on the apical side of the epithelial cells; and
measuring a parameter of the administration of the compound and optionally the immune cells, such as the peripheral blood mononuclear cells.

2. The method of claim 1, wherein the organ chip is an intestine chip, wherein the polarized epithelial cells are intestine epithelial cells.

3. The method of claim 2, wherein the polarized epithelial cells comprise Caco-2 or primary cells, such as primary human intestinal epithelial cells.

4. The method of claim 1, wherein the organ chip is a lung chip, wherein the polarized epithelial cells are alveolar epithelial cells or primary cells.

5. The method of claim 4, wherein the basal channel comprises pulmonary microvascular endothelial cells.

6. The method of claim 1, wherein determining the localization and optionally the expression level of the target on the polarized epithelial cells is performed by immunofluorescence analysis.

7. The method of claim 1, wherein determining the localization and optionally the expression level of the target on the polarized epithelial cells comprises comparing the localized target on the polarized epithelial cells with the target in a native organ, such as in native intestine or in native lung.

8. The method of claim 1, wherein the compound is a compound to be administered via systemic circulation.

9. The method of claim 1, wherein the polarized epithelial cells and/or the basal channel and/or the apical channel is covered with an extracellular matrix.

10. The method of claim 1, wherein the target on the polarized epithelial cells is an antigen, such as a tumor-overexpressing protein.

11. The method of claim 1, comprising administering the compound to the basal channel and then measuring a parameter of the administration of the compound and the peripheral blood mononuclear cells, and further comprising:
administering the compound and peripheral blood mononuclear cells to the apical channel; and
measuring a parameter of the administration of the compound and optionally the peripheral blood mononuclear cells.

12. The method of claim 1, wherein the parameter is:
a safety parameter associated with a state of the polarized epithelial cells and/or with a state of a tight barrier formed by the polarized epithelial cells; and/or
pharmacological parameter.

13. The method of claim 12, wherein the parameter is a safety parameter associated with the integrity or permeability of the barrier formed by the polarized epithelial cells and/or a pharmacological parameter associated with side effects or potency of the compound, peripheral blood mononuclear cell activation or cytokine release.

14. The method of claim 1, wherein the flow rate of the administered compound and optionally the immune cells, through the apical channel or the basal channel is 10 to 60 μl/h, 15 to 50 μl/h, 20 to 40 μl/h, or essentially 30 μl/h.

15. The method of claim 1, wherein the compound is an antibody, a bispecific antibody, such as a T-cell bispecific antibody, or a small molecule.

16. The method of claim 15, wherein the compound is a bispecific antibody, such as a T-cell bispecific antibody, that binds an epithelial target and an immune cell.

17. The method of claim 1, wherein the organ chip comprises a first and a second vacuum channel, wherein the main channel is arranged between the first and second vacuum channel, wherein peristaltic movement is modelled by the first and second channels during the administering of the compound and optionally immune cells, such as peripheral blood mononuclear cells (PBMC) and/or during the measuring of the parameter of the administration of the compound and optionally the immune cells, such as the peripheral blood mononuclear cells.

18. The method of claim 1, first performed with a first compound of interest and thereafter again performed with a second compound of interest, and further comprising comparing the parameter for the first compound of interest and the parameter for the second compound of interest are compared.
